(19) United States
(12) Patent Application Publication
(10) Pub. No.: US 2003/0220696 A1
(43) Pub. Date: Nov. 27, 2003

(54) Implantable porous metal

(76) Inventors: David Jerome Levine, Cincinnati, OH (US); Donald Warren Graham, Cincinnati, OH (US); Jeffrey Ewald Anto, Cincinnati, OH (US)

Correspondence Address: Beverly A. Lyman, Ph.D., Wood, Herron & Evans, L.L.P., 2700 Carew Tower, 441 Vine Street, Cincinnati, OH, 45202-2917, US

(21) Appl. No.: 10/443,209

(22) Filed: May 22, 2003

Related U.S. Application Data
(60) Provisional application No. 60/382,769, filed on May 23, 2002.
(60) Provisional application No. 60/385,177, filed on May 31, 2002.

(51) Int. Cl. A61F 2/28

(52) U.S. Cl. 623/17.17; 623/23.55; 623/23.76; 607/51

(57) ABSTRACT

An implantable composition of a biocompatible porous metal for enhanced tissue in-growth and fixation in the body. The metal has a porosity greater than 80% and up to about 95% which allows good cell population, yet it also provides structural integrity and stability allowing its use as a weight-bearing implant. In various embodiments, the metal may be titanium, which includes titanium alloys, or may be a cobalt-chromium-molybdenum alloy. The high porosity desirably facilitates in-growth of cells and/or tissues, which in turn facilitates biological fixation and biocompatibility. This is beneficial, for example, in an orthopedic implant such as a hip replacement, for facilitating in-growth of connective tissue and bone cells. The porous composition is structurally stable.

### RELATED APPLICATION

This application claims priority to Provisional application, U.S. Application Serial No. 60/382,769 filed May 23, 2002, now pending, and to Provisional application, U.S. Application Serial No. 60/385,177 filed May 31, 2002, now pending.

### FIELD OF THE INVENTION

The invention is directed to biological implants, or coatings for such implants, of porous metal structures supporting tissue or cellular in-growth.

### BACKGROUND OF THE INVENTION

Metal implants or prostheses provide structure and support when surgically implanted. For example, hip or knee weight-bearing implants may permit a non-ambulatory patient to walk, or may permit greater mobility to a patient with limited mobility.

Many implant compositions are available. The technology for the fabrication of implants coated with a porous surface made from spherical powders has been available for many years. For example, orthopedic implants are known which have cobalt-chromium-molybdenum (Co—Cr—Mo) or titanium porous surfaces, manufactured using spherical powders. These types of porous coated implants have been widely used in hip stems, femurs, tibias, shoulders, elbows, fingers, etc.

The process for the fabrication of implants coated with a beaded structure presenting a porous surface necessitates thermal exposure of the implant at elevated temperatures for periods of one-half hour to four hours. The resultant coating layer has a porosity of about 35% and a density of about 65%. The pore sizes of the coating can be controlled by selecting the powder particle sizes for optimum biological fixation. Typical pore sizes specified for porous coatings of implant devices range from 50 μm to 500 μm.

Porous structures fabricated from spherical powder particles or beads by a gravity sintered process have been used to create such porous coatings on implant substrates. In small diameter cross-sections, however, these structures were not sufficiently strong and stable for use as bone repair scaffolding, nor were they suitable for soft tissue attachments.

The greater the degree of porosity in the implant, the greater extent that cells and tissues can fill the pores and help to anchor and stabilize the implant in the body. This need for enhanced porosity of an implant has been recognized, for example, U.S. Pat. No. 6,312,473 discloses that 80% void, 100-500 μm diameter, and inter-pore connections (100-200 μm) for tissue in-growth are consistent with appropriate bulk mechanical properties (ultimate tensile strength 1 MPa) for an orthopedic implant. The '473 patent discloses that the average pore size is in the range of 10-500 μm, with pore sizes less than 10 μm having surfaces which exhibit toxicity to cells, and pore sizes greater than 500 μm resulting in surfaces which lack sufficient structural integrity.

Thus, there must be a balance between the amount, type, porosity, etc. of the metal needed to provide the required degree of structural support to the implant, and the extent of porosity so that sufficient cell growth can be achieved and maintained in and around the implant. Implants with improved porosity to provide supports that are not toxic to cells and have the desired structural integrity and stability, yet also maximize the available area for tissue or cellular in-growth beyond that which is presently available are desirable, but are at present not available using spherical powders.

### SUMMARY OF THE INVENTION

The invention discloses an implantable porous metal meeting the needs for enhanced porosity and yet also providing sufficient structural integrity. In one embodiment, the invention is directed to a biocompatible porous metal three-dimensional structure having a porosity greater than 80% and up to about 95% capable of enhanced tissue in-growth yet sufficiently stable and non-fragile to provide structural integrity. The metal may be titanium and/or titanium alloys such as a titanium-niobium alloy, or a cobalt-chromium-molybdenum (Co—Cr—Mo) alloy. The structure has a tensile strength of at least 5000 psi. The implant may be inoculated with or may contain cells or tissues, and/or at least one biological agent such as a drug, a protein, a peptide, a peptide fragment, etc. It may be pre-inoculated with these cells or biological agents before surgical implantation, and/or may become populated with cells after implantation.

In another embodiment, the invention is directed to the above described porous metal structure and at least one cell on at least one surface of the implant. The cell may be a bone cell such as an osteoblast, osteocyte, or osteoclast as would be useful for a hip implant, a knee implant, shoulder implant, elbow implant, finger implant, mandibular implant, etc. In a particular embodiment of a mandibular implant, the porous metal may be shaped to fit in the gum to augment a bony support needed to anchor dentures. The cell in the implant may also be a muscle cell, a nerve cell, a skin cell, a blood cell, etc., as would be useful for an implant at an excision site where a tumor has been surgically excised and, where additional innervation, or vascularization, blood supply, skin growth, muscle function, etc. is desired. The cell may be genetically modified, and/or organized to form a tissue, such as connective tissue and/or fibrous tissue. Combinations of cell types in the implant may be used, for example, bone cells to provide a bony scaffold and endothelial cells to form blood vessels to vascularize and nourish this bony scaffold.

In other embodiments, the invention may be used to treat a tumor when the porous metal structure is implanted in or near a tumor site. The structure contains antineoplastic agents, and/or is targeted with radiant energy sufficient to increase the temperature of the metal implant to effect thermal tumor therapy.

In another embodiment, the invention is directed to treating a patient with the above described porous metal structure. The porous biocompatible metal is implanted in the patient and cellular in-growth is facilitated. In one embodiment, the porous metal structure is freestanding. In another embodiment, the porous metal structure is attached to a prosthetic implant, for example by sintering or gluing to the implant. In still another embodiment, the porous metal structure is created on a prosthetic implant. The porous metal structure may contain the cell, tissue, biologic agent, etc., before or after implantation.

These and other aspects of the invention will be apparent with reference to the following figures, description, and examples.

### BRIEF DESCRIPTION OF THE FIGURES

FIG. 1 is a perspective view of a freestanding embodiment of the porous metal structure.

FIG. 2 is a perspective view of an embodiment of the porous structure for use with a prosthetic implant.

FIG. 3 is a detailed view of the porous structure in an implantable device and showing tissue in-growth.

FIG. 4 shows the porous structure implanted in a mandible supporting bone cell in-growth.

### DETAILED DESCRIPTION

Implantable biocompatible structures (implants) having a porosity in the range of about 80% to about 95% permit biological fixation with the host tissue or structure and enhance tissue in-growth within openings defined by pores in the structures. Such porosity is desirable because a large number of fixation points are achieved due to the enhanced extent of in-growth. Transmitted loads are thus distributed over a larger area than with less porous structures, thereby minimizing the stress applied to the interface between the host tissue and the implant.

The invention contemplates the use of an implantable biocompatible porous metal three-dimensional structure that does not require the use of spherical powders or beads for its manufacture and which facilitates enhanced tissue in-growth. As used herein, the biocompatible porous metal includes sinterable ceramics, elemental metals, and alloys which now exist or which may be developed in the future having an interconnected porosity greater than 80% and up to about 95%. An interconnected porosity indicates that all pores are connected either directly or indirectly, and there are no closed cavities. The implantable structure may be freestanding. Alternatively, the implantable structure may be fabricated directly with a prosthesis or implant, or it may be coated on an implant.

One embodiment of the invention uses metal structures of titanium and/or a titanium alloy. Titanium includes unalloyed commercially pure titanium (CPTi; ASTM F 67) and wrought titanium alloys or cast titanium alloys such as Ti-6Al-4V (ASTM F 136). Titanium alloys also include a titanium-niobium alloy having about 5% niobium to about 25% niobium. Another embodiment of the invention uses a cobalt-chromium-molybdenum alloy (Co—Cr—Mo, ASTM F 75). The above-described metals and alloys have an interconnected porosity greater than 80% and up to about 95%, and are available from commercial sources, including AstroMet, Inc. (Cincinnati Ohio), TiCoMET Engineering Co. (Cincinnati Ohio), and from Porvair (Hendersonville N.C.) for non-medical uses. Alternatively, one way of manufacturing the structure is by a replicating process utilizing a urethane precursor in a range of pore sizes. The urethane is burned off leaving a metal structure behind. Any other suitable manufacturing process can be used to result in a structure of titanium and/or titanium alloy or a Co—Cr—Mo alloy having a porosity greater than 80% and up to about 95%.

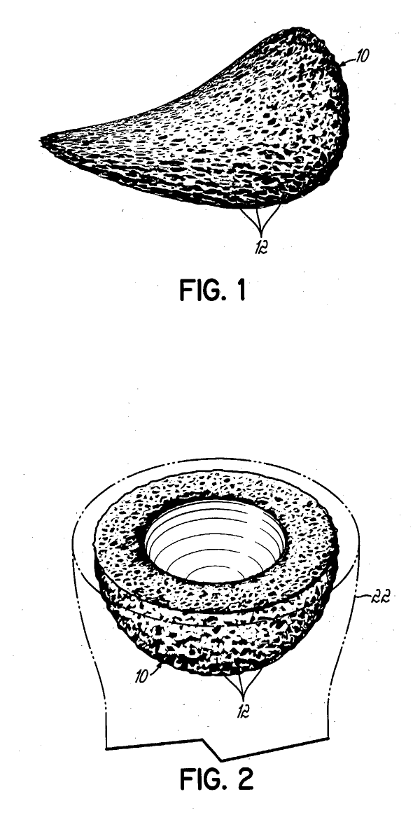

One embodiment of the invention, as shown in FIG. 1, is a porous metal structure 10 used as a freestanding scaffold for tissue repair, such as bone repair. The structure may be in bulk shape, or may be in a desired shape, for example, to fit a small implant site such as a finger or mandible, or to be contoured to a desired topography of an anatomical site.

Another embodiment of the invention, as shown in FIG. 2, is a porous metal structure 10 as part of a prosthetic implant 22, for example. The porous metal structure 10 may be coated or provided on the implant 22 by several methods. It may be attached to the implant 22, for example, by sintering or gluing using a biocompatible glue such as polymethyl methacrylate (Stryker, Rutherford N.J.) to achieve a bond capable of withstanding up to about 7000 psi stress. It may be created on the implant 22, for example, by coating the implant with a polyurethane precursor and binder, drying, providing the metal powder, and thereafter curing. In any of the above-described embodiments, the depth of the porous metal coating may be in the range of about 2 mm to about 5 mm.

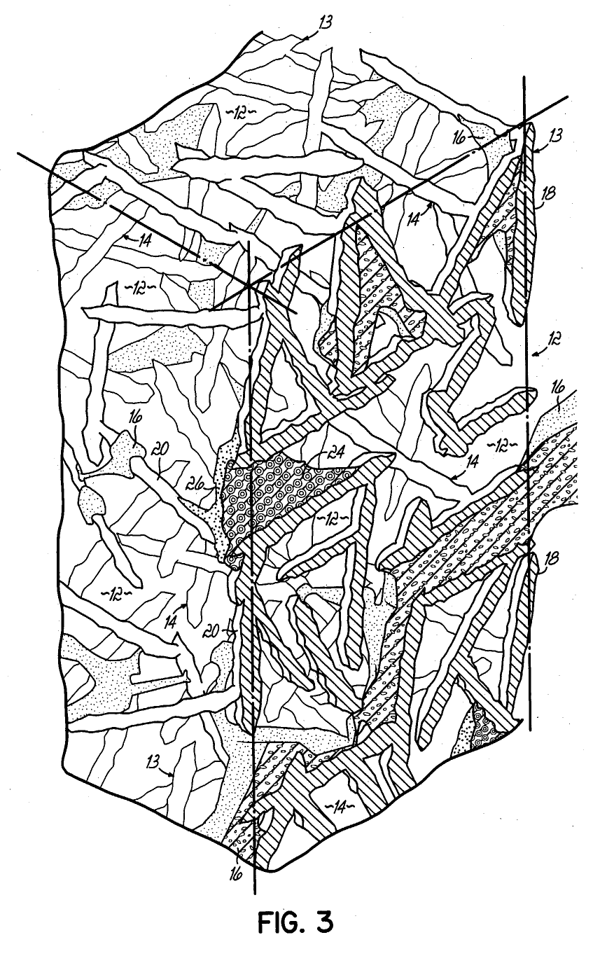

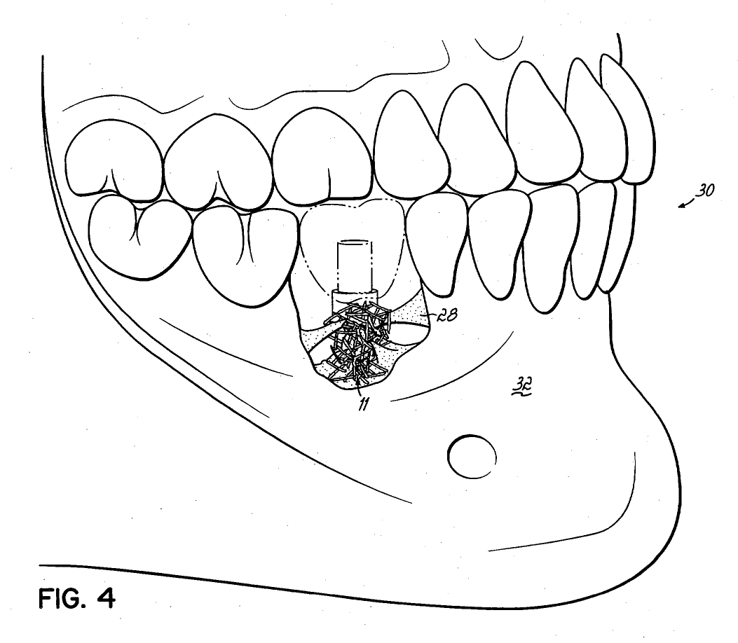

As shown in FIG. 3, the metal 13 defines pores 12. The pores 12 have internal surfaces 18, external surfaces 20, and interfacial surfaces or interstices 14 which permit, and provide a scaffold for, the in-growth of cells and/or tissues 16. As such, a trabecular structure is formed, similar to those that occur naturally with supporting strands of connective tissue projecting into an organ and constituting part of the framework of that organ.

In all the above embodiments, the 80-95% porosity structure 10 enhances in-growth of biological material, whether the biological material is pre-inoculated on the structure before surgical implantation (e.g., in vitro, ex vivo), or whether the biological material is supplied in vivo. The biological material may be a cell and/or tissue 16 supported in and/or on the porous structure 10, for example, for in-growth of soft tissue, for tendon attachment, etc. The cells and/or tissues may be obtained from commercial sources, such as commercially available cell lines from the American Type Culture Collection (ATCC, Manassas Va.). Cells and/or tissues may be from biological sources, for example, the implant recipient, in which case the implant is an autologous structure, or another human, in which case the implant is an allogeneic structure, or another species, in which case the implant is a xenogeneic structure, or the cells/tissues may be from multiple sources, in which case the implant is a chimeric structure. The biological material may also include a vehicle 24, such as a microcapsule or microparticle, containing an agent such as a pharmaceutic to deliver or provide the agent to the area of implant or to the surrounding area for preventative, therapeutic, and/or diagnostic purposes. The biological material may be a natural or synthetic nucleic acid, protein, peptide, and/or peptide fragment, and may contain a targeting agent such as an antibody or antigen.

The structure 10 having open, interconnected pores 12 has a density of less than 100% of theoretical. Porosity and pore sizes are measured as known to one skilled in the art, such as by metallographic or stereological methods. In one embodiment, the structure has a density of about 10% of theoretical, rendering about 90% of its volume available for tissue in-growth. In another embodiment, the porous metal has a density less than 15% to about 5% of theoretical, rendering greater than 80% and up to about 95% of its volume available for tissue ingrowth. In contrast, the porous coatings made from spherical powders and presently used for bone in-growth and fixation have a porosity that is only about 35% of theoretical, rendering only about 65% of its volume available for tissue in-growth.

While available in-growth volume is the mathematically calculated percentage, investigators have determined that the degree of filling the internal coating porosity by bone is estimated to average generally between 30% and 50%. Therefore, because the porosity of the coating previously available is only about 35%, the total interfacial surface of bone fixation is likely to be between about 10% to about 17.5%. In contrast, the porosity of the inventive structure is greater than 80% porous, and may be up to about 95% porous. Thus, the interfacial area available for tissue in-growth, for example bone in-growth, is in the range of about 24% to about 40%, and may be up to about 48%. This is almost triple that of the previously available beaded coating structure.

The increased porosity and range of pore sizes in the structure 10 permit enhanced fixation characteristics upon surgical implantation, in comparison to the presently available porous coating systems made with spherical powders. Moreover, the morphology of the inventive structure 10 mimics the natural formation of cancellous bone. Cancellous bone consists of a three-dimensional lattice of branching bony spicules or trabeculae delimiting a labyrinthine system of intercommunicating spaces that are occupied by bone marrow in vivo. The textured morphology and the interstitial network of the inventive structure 10, therefore, is more adaptable to support and/or stimulate tissue in-growth, such as bone in-growth.

Besides good in-growth and good fixation, the structure 10 provides good delivery of pharmaceutical agents or other agents contained therein. In one embodiment, the structure 10 may be seeded with vehicles 24 or vesicles containing an agent or a drug 26, such as antineoplastic drugs, and the structure 10 may be implanted in or near a tumor site for localized delivery of the antineoplastic drug. An example is an implant in a bone containing osteoblasts and osteocytes and at least one antineoplastic drug to target an osteosarcoma. Other drugs or agents 26, such as therapeutic agents or diagnostic agents, may also be used. The porous metal structure to be implanted may be pre-inoculated with one or more cell or agent, and/or it may be populated with cells and/or dosed with agents after implantation.

In another embodiment, the structure 10 is provided with cells and/or tissues 16, with tissues being a higher organization of one or more cell types. As known to one skilled in the art, such inoculation or seeding with cells or tissues can be provided in vivo or in vitro, using techniques known to one skilled in the art. The cells may be immature cells or cell precursors such as stem cells, and/or mature cells. The cells may be in any state, such as quiescent, dividing, senescent, etc. The cells may be genetically engineered and/or may be recombinant cells. The cells may be bone cells, such as osteoblasts, osteocytes, and/or osteoclasts. The cells may be muscle cells (myocytes), including smooth, striated, and/or cardiac muscle cells. The cells may be nerve cells (neurons). The cells may be skin cells, for example, epidermal cells such as epithelial cells, keratinocytes, melanocytes, immunocytes, and/or stem cells, dermal cells such as fibroblasts, corneocytes, melanocytes, etc. The cells may be blood cells including hematopoietic stem cells, leukocytes, platelets, megakaryocytes, histiocytes, plasma cells, mast cells, fibroblasts, etc. Tissues may include structural tissues such as connective tissue, fibrous tissue, soft tissue, skin, etc.

In particular embodiments, the porous metal structure 10 may be implanted at a site from which a tumor had been removed to provide tissue in-growth at these sites. For these purposes, the strength requirements for the porous metal structure are less relevant because there is little or no weight being applied. Rather than providing a mechanical support, in these embodiments the structure provides a space-filling support to facilitate tissue filling, permeation, and in-growth in cavities previously occupied by a tumor. For example, the structure may be implanted in the leg at a site in which a solid tumor was removed. It may be desirable to enhance muscle cell in-growth, in which case the porous metal structure may be seeded with myocytes, as well as provide attachment to existing tissue, in which case connective tissue precursors may also be provided, etc. Other examples will be appreciated by those skilled in the art.

The cells and/or tissues 16 may be seeded and/or applied to or in the structure in vitro in any combination under conditions facilitating cell maintenance and growth. These conditions include regulation of appropriate temperature, humidity, O₂/CO₂ saturation, incubation in media containing amino acids, peptides, proteins, inorganic salts, carbohydrates, vitamins, serum, growth factors, cytokines, hormones, nutrients, supplements, etc., as appropriate for the particular cell type or types as known to one skilled in the art, for example, and as disclosed in T. Maniatis et al., Molecular Cloning: A Laboratory Manual, Cold Spring Harbor Press, 1982; and J. M. Davis, Basic Cell Culture: A Practical Approach, second edition, 2002, the relevant sections of which are incorporated by reference herein. Media are available from commercial sources, for example, Sigma-Aldrich Products for Life Science Research 2001 (St. Louis Mo.).

In one embodiment and with reference to FIG. 4, the porous metal structure 10 may be implanted in the mandible 32 of the jaw 30 to promote bone thickening. After wearing dentures for many years, most patients suffer extensive mandibular bone loss due to atrophy. The shriveling of the mandible causes loosening of the dentures and, in many cases, it becomes impossible for a dentist to make properly fitting new dentures because very little bone remains to hold the new dentures in place.

To correct this problem, most dentists recommend dental implants. Atrophied mandibular bone, however, is too thin to support dental implants, and methods to increase the thickness of the mandible must be used. One method involves implanting a porous hydroxylapatite (HA) (ASTM F 1185) ceramic under the gum tissue. HA has a chemical composition similar to bone and tends to induce new bone to generate and grow into the porous structure. Eventually the HA dissolves and is replaced by new bone. After several months, enough new bone is generated to support a dental implant. After a few more months, the implant is exposed and an abutment is placed on the implant and is capped.

In this embodiment of the invention, the previously described porous metal structure 10 is used either along with, or in place of, HA as a support for growth of new bone. More specifically, the porous structure 10 can be used“as is”, or may contain HA and/or cells 28, such as osteoblasts or osteocytes, tissues, a biologic agent, etc., to enhance new mandibular bone growth. The bony structure then remains implanted in the mandible 32 to provide stability and strength to the patient's existing natural bone. The porosity of the structure facilitates the rate and extent of mandibular bone generation, and causes less trauma to the patient than with currently available structures. The structure may be also fitted to the patient's existing mandibular topography to optimize strength and stability of the support.

In another embodiment, the structure 10 contains a diagnostic agent and/or a therapeutic agent 26. The structure 10 may be implanted at a specific site or may be implanted at a generalized site and contain targeting agents such as an antibody or antigen to attract desired cells or cellular components.

In still another embodiment, the structure 10 may be used in tumor therapy, either in addition to or in place of the previously described embodiment wherein antineoplastic drugs are contained with the structure. In this embodiment, the implanted structure 10 is subjected to localized radiant energy sufficient to effect tumor destruction by heat, that is, thermal therapy. It has been reported that some malignant tumors can be reduced or completely eliminated with thermal therapy. However, non-localized thermal therapy, wherein the body temperature is increased over its normal temperature of 98.6° F. and is up to 107° F. for a period of time is not without risk to the patient.

As an alternate to non-localized thermal tumor therapy, a method using the inventive porous metal structure 10 to localize thermal therapy is provided. The porous metal structure 10 is implanted at or near the tumor site. A penetrating energy beam (e.g., X-ray, gamma-ray, microwave, etc.) is then focused at the implant/tumor site, for example, by using a laser, as known by one skilled in the art. The specific implant material and energy wavelength are matched to result in a localized increased temperature of the implant in the range between normal temperature and up to about 107° F. The exact conditions (e.g., temperature, energy, duration, etc.) may be determined by one skilled in the art. For example, the specific heat capacity of Ti and F-75 (Co) is 0.125 and 0.101, respectively. At 300° K., the quantity of heat needed to produce a unit change in temperature can be determined by one skilled in the art, and is expressed as calories/g/° C. The exposure and frequency of treatments are also adjusted to increase the likelihood of eliminating or reducing the size of the tumor, or preventing further growth of the tumor. The implanted structure allows thermal therapy to be repeated as often as desired or necessary.

The invention will be further appreciated with reference to the following example.

### EXAMPLE

A freestanding three-dimensional structure having dimensions of 30 mm×20 mm×6 mm is implanted into dorsal subcutaneous tissue of an anesthetized large breed canine. Using a posterior approach, a soft tissue pocket is created between the subcutaneous fat and fascia. Two pockets are created and the structure is sutured in each pocket and irrigated with sterile saline. The wound is closed. The animals are permitted to recover and the structures are retrieved from the animal after 4 weeks, 8 weeks, and 16 weeks by dissecting out with a flap (4 cm) of the overlying tissue.

The implant/tissue is used in mechanical testing. The extent of tissue in-growth is determined, for example, by either tension or push-out tests using a tensile testing apparatus such as that available from Tinius-Olsen or Amatek. The tissue is prepared histologically and is examined microscopically to qualitatively and quantitatively assess the in-grown tissue, for example, its nature, vascularity, extent, etc.

Other variations or embodiments of the invention will also be apparent to one of ordinary skill in the art from the above description. Thus, the foregoing embodiments are not to be construed as limiting the scope of this invention.

What is claimed is:

1. An implantable device comprising a biocompatible metal having a porosity greater than 80% up to about 95% and selected from the group consisting of titanium, a titanium alloy, and a cobalt-chromium-molybdenum alloy, capable of supporting tissue in-growth.

2. The device of claim 1 wherein the porosity is at least 90%.

3. The device of claim 1 further comprising at least one biologic agent selected from the group consisting of a cell, a tissue, a pharmaceutical and combinations thereof on at least one surface of the metal.

4. The device of claim 3 wherein the biological agent is in a matrix selected from the group consisting of a biocompatible polymer, a biocompatible vesicle, a microcapsule, a microparticle, a liposome, and combinations thereof.

5. The device of claim 3 wherein the biological agent is a diagnostic agent.

6. The device of claim 3 wherein the biological agent is a therapeutic agent.

7. The device of claim 3 wherein the biological agent contains a targeting compound.

8. The device of claim 1 for implanting at an anatomical site selected from the group consisting of a hip, a shoulder, a knee, a finger, an elbow, a mandible, and combinations thereof.

9. An implantable device comprising a structure of a biocompatible metal selected from the group consisting of titanium, a titanium alloy, and a cobalt-chromium-molybdenum alloy and having a porosity greater than 80% and up to about 95% and at least one cell capable of at least about 24% in-growth in the device.

10. The device of claim 9 wherein the cell is selected from the group consisting of a quiescent cell, a dividing cell, a senescent cell, an immature cell, a cell precursor, a stem cell, and combinations thereof.

11. The device of claim 9 wherein the cell is selected from the group consisting of a bone cell, a muscle cell, a nerve cell, a skin cell, an epithelial cell, a blood cell, and combinations thereof.

12. The device of claim 9 wherein the cell is selected from the group consisting of an osteoblast, an osteocyte, an osteoclast, an erythrocyte, a leukocyte, a platelet, a megakaryocyte, a histiocyte, a plasma cell, a mast cell, a fibroblast, and combinations thereof.

13. The device of claim 9 wherein the cell comprises a tissue.

14. The device of claim 13 wherein the tissue is selected from the group consisting of connective tissue, fibrous tissue, blood, and combinations thereof.

15. An implantable device comprising a structure of a biocompatible metal selected from the group consisting of titanium, a titanium alloy, and a cobalt-chromium-molybdenum alloy and having a porosity greater than 80% and up to about 95% and at least one cell filling at least about 24% of the porosity in the device.

16. An implantable structure comprising a biocompatible metal having a porosity greater than 80% and up to about 95% and selected from the group consisting of titanium, a titanium alloy, and a cobalt-chromium-molybdenum alloy, and at least one biological agent selected from the group consisting of a cell, a non-cell biologic agent, and combinations thereof, the structure attached to an implant.

17. The structure of claim 16 attached by sintering to the implant.

18. The structure of claim 16 attached by gluing to the implant.

19. An implantable structure comprising a biocompatible metal having a porosity greater than 80% and up to about 95% and selected from the group consisting of titanium, a titanium alloy, and a cobalt-chromium-molybdenum alloy, and at least one biological agent selected from the group consisting of a cell, a non-cell biologic agent, and combinations thereof, the structure fabricated on an implant.

20. An implantable structure comprising a biocompatible metal having a porosity greater than 80% and up to about 95% and selected from the group consisting of titanium, a titanium alloy, and a cobalt-chromium-molybdenum alloy, and at least one biological agent selected from the group consisting of a cell, a non-cell biologic agent, and combinations thereof, the structure shaped to fit an implant site.

21. A therapeutic method comprising
implanting a device comprising a biocompatible metal with pores having a porosity greater than 80% up to about 95% and selected from the group consisting of titanium, a titanium alloy, and a cobalt-chromium-molybdenum alloy, the device capable of supporting tissue in-growth, and
enhancing cell in-growth in said pores.

22. The method of claim 21 producing at least 24% cell in-growth.

23. A method to enhance mandibular bone regeneration comprising
(a) implanting in a mandible a biocompatible porous metal structure having greater than 80% and up to about 95% porosity, the metal selected from the group consisting of titanium and a cobalt-chromium-molybdenum alloy, and
(b) attaching the implanted structure to the patient's mandible to enhance bone in-growth in the porous structure.

24. The method of claim 23 wherein the implanted structure further comprises hydroxylapatite.

25. The method of claim 23 wherein the mandible has a surface topography and the structure is shaped to the surface topography.

26. The method of claim 23 wherein the structure is attached to the patient's mandible by screwing or stapling.

27. An implantable device for localized thermal tumor therapy in a patient comprising
(a) implanting at a tumor site the device comprising a biocompatible porous metal structure having greater than 80% and up to about 95% porosity, the metal selected from the group consisting of titanium, a titanium alloy, and a cobalt-chromium-molybdenum alloy, and
(b) increasing the temperature of the implant for a duration to thermally treat the tumor with radiant energy to the implanted structure.

28. The method of claim 27 wherein the energy is selected from the group consisting of x-rays, gamma-rays, microwaves, and combinations thereof.

29. The method of claim 27 wherein the temperature is increased to result in a temperature of the implant in the range greater than 98.6° F. and up to 107° F.

30. The method of claim 27 further comprising repeating step (b) at a desired treatment interval.

31. The method of claim 27 wherein a laser provides radiant energy.

32. A method of cell in-growth in an implantable structure comprising
(a) inoculating a cell on a biocompatible metal structure having a porosity greater than 80% and up to about 95%, the metal selected from the group consisting of titanium, a titanium alloy, and a cobalt-chromium-molybdenum alloy, and
(b) providing culture conditions to the inoculated structure to obtain cell in-growth of at least 24%.

33. The method of claim 32 wherein the cell in-growth is at least 27%.

34. The method of claim 32 wherein the cell in-growth is in the range of 24% and up to about 48%.

35. The method of claim 32 wherein culture conditions are selected from the group consisting of nutrient media, temperature, O₂/CO₂ saturation, supplements, and combinations thereof.

36. The method of claim 32 wherein the cell is selected from the group consisting of a quiescent cell, a dividing cell, a senescent cell, an immature cell, a cell precursor, a stem cell, and combinations thereof.

37. The method of claim 32 wherein the cell is selected from the group consisting of a bone cell, a muscle cell, a nerve cell, a skin cell, an epithelial cell, a blood cell, and combinations thereof.

38. The method of claim 32 wherein the cell is selected from the group consisting of an osteoblast, an osteocyte, an osteoclast, an erythrocyte, a leukocyte, a platelet, a megakaryocyte, a histiocyte, a plasma cell, a mast cell, a fibroblast, and combinations thereof.

39. The method of claim 32 wherein the cell comprises a tissue.

40. The method of claim 32 wherein inoculation occurs in vivo.

41. The method of claim 32 wherein inoculation occurs in vitro.

42. An implantable composition comprising at least one biological agent and a biocompatible sinterable material having a porosity greater than 80% up to about 95%.

43. The composition of claim 42 wherein the biocompatible sinterable material is selected from the group consisting of an elemental metal, an alloy, and a ceramic.

44. The composition of claim 42 wherein the biocompatible sinterable material is selected from the group consisting of titanium, a titanium alloy, and a cobalt-chromium-molybdenum alloy.

45. An article comprising an implantable metal structure having interconnected pores to provide a porosity greater than about 80% up to about 95%, a density less than 15% of theoretical, and a tensile strength of at least 5000 psi, the pores defining an interfacial surface capable of supporting tissue growth into the structure.

46. The article of claim 45 on a device capable of implantation in a mammal.

47. The article of claim 45 on a prosthesis.

48. The article of claim 45 further comprising at least one therapeutic agent.

49. An article comprising a porous metal selected from the group consisting of titanium, a titanium alloy, and a Cobalt-Chromium-Molybdenum alloy, the metal formed into a reticulated structure having at least 80% and up to 95% interconnected pores, the structure having a tensile strength of at least 5000 psi.

50. The article of claim 49 as a freestanding implant.

51. The article of claim 49 on an implantable device.

52. A reconstructive method comprising implanting in a patient at a site requiring tissue replenishment under replenishment facilitating conditions a structure of a metal selected from the group consisting of titanium, a titanium alloy, a cobalt-chromium-molybdenum alloy, the structure having an interconnected porosity greater than about 80% up to about 95%, a theoretical density less than 15%, and a tensile strength of at least 5000 psi, the pores defining an interfacial surface for in-growth of tissue into the structure thereby replenishing tissue at the site.

53. The method of claim 52 replenishing atrophied bone.

54. The method of claim 52 where the structure is implanted in a mandible.

55. The method of claim 52 replenishing tissue at a site from which a tumor was removed.
